(19) United States
(12) Patent Application Publication
(10) Pub. No.: US 2025/0000363 A1
(43) Pub. Date: Jan. 2, 2025

(54) SENSOR MODULE FOR VITAL SIGN MONITORING AND METHOD

(71) Applicant: ams-OSRAM International GmbH, Regensburg (DE)

(72) Inventors: Zeljko PAJKIC, Regensburg (DE); Johann RAMCHEN, Hausen (DE); Michael MUELLER, Pfaffenhofen (DE)

(21) Appl. No.: 18/711,092

(22) PCT Filed: Nov. 17, 2022
(86) PCT No.: PCT/EP2022/082342

§ 371 Date: May 16, 2024

(30) Foreign Application Priority Data
Nov. 19, 2021 (DE) 10 2021 130 371.0

(51) Int. Cl. A61B 5/00

(52) U.S. Cl. CPC A61B 5/0059; A61B 5/681; A61B 5/742

(57) ABSTRACT

A sensor module, in particular for detecting a vital sign parameter, includes a semiconductor body with a first main surface and a second main surface opposite the first main surface. The sensor module also includes an integrated circuit integrated in the semiconductor body, and a photodetector integrated in the semiconductor body and arranged on the first main surface. The photodetector is controllable by means of the integrated circuit. The sensor module further includes a plurality of contact elements on the first main surface of the semiconductor body. The plurality of contact elements are electrically connected to the integrated circuit. The sensor module additionally includes at least one μLED arranged on two of the plurality of contact elements and electrically connected thereto.

The present application claims the priority of German patent application No. 10 2021 130 371.0 dated Nov. 19, 2021, the disclosure of which is hereby incorporated by reference into the present application.

A sensor module is specified, in particular for detecting a vital parameter. In addition, a use of the sensor module is specified.

### BACKGROUND

The measurement of vital parameters or vital functions (VSM: vital sign monitoring) such as heart rate, heart rate variability or oxygen content in the blood can be carried out using a PPG (photoplethysmogram), for example. A photoplethysmogram is an optically obtained plethysmogram that can be used to record blood volume changes in the microvascular tissue bed. A PPG is often obtained with a pulse oximeter that illuminates the skin and measures changes in light absorption based on the light reflected back from the skin. The reflected-back signal detected by a photodiode over the time t consists of light reflections from the tissue or skin, light reflections from the blood in the veins, which is particularly poor in oxygen, and light reflections from the blood in the arteries, which is particularly rich in oxygen. This signal can then be used to obtain information about, for example, heart rate or oxygen content.

As shown in FIG. 1, the components B required for such a measurement currently consist of several discrete emitters E₁, E₂, E₃ and detectors D, which are arranged in a so-called “side-by-side” design, i.e. next to each other, in individual cavities K provided for this purpose. However, this type of arrangement means that the components B usually require a lot of space and are usually expensive to manufacture due to the required backend materials and associated manufacturing processes.

However, due to the ever-increasing complexity and simultaneous miniaturization of wearable devices such as smartphones, chest straps for fitness trackers, smartwatches or fitness wristbands, it is necessary that the components within such a device, which are required to record a vital parameter, are also smaller or more compact.

There is therefore a need to specify a sensor module, in particular for detecting a vital parameter, which works reliably, has a high measurement accuracy and is compact in design.

### SUMMARY OF THE INVENTION

This need is met by a sensor module mentioned in claim 1. Claim 15 specifies the features of a use of such a sensor module according to the invention. Further embodiments are the subject of the subclaims.

The core idea of the invention is to integrate both the emitter and the detector(s), which are required to detect a vital parameter, on or in an integrated circuit. On the one hand, this eliminates the need for a separate substrate on which the components have to be mounted, and on the other hand, complex cabling of the individual components is no longer necessary. In addition, μLEDs, i.e. very small LEDs, are used as emitters so that the overall component can be very compact, not least due to the small emitters. In particular, the integration of μLEDs on the integrated circuit in the form of unhoused semiconductor chips, which are built up in the wafer-level process, can save both space and, in particular, costs due to the elimination of back-end materials.

Unhoused may mean that the semiconductor chip has no package around its semiconductor layers, such as a “chip die”. In some embodiments, unhoused may also mean that the semiconductor chip is packaged using wafer-level packaging (WLP) while still part of a wafer. A resulting “package” has practically the same size as the semiconductor chip, which is why this case can also be referred to as an unhoused semiconductor chip.

A micro LED, also known as a μLED or μLED chip, is in particular a very small LED (light emitting diode) whose edge lengths are in the range of 100 μm to 10 μm. For example, a μLED can have a spatial dimension of 50 μm×50 μm, or for example 100 μm×100 μm.

According to one embodiment, a sensor module, in particular for detecting a vital parameter, comprises a semiconductor body, in particular a silicon semiconductor body, with a first main surface and a second main surface opposite the first. An integrated circuit (IC), in particular a silicon driver IC, is integrated into the semiconductor body. The sensor module also comprises a photodetector integrated into the semiconductor body and arranged on the first main surface, which can be controlled by means of the integrated circuit. A plurality of contact elements, which are electrically connected to the integrated circuit, are arranged on the first main surface of the semiconductor body. Furthermore, the sensor module comprises at least one LED, wherein the μLED is arranged on two of the plurality of contact elements and is electrically connected to them.

According to at least one embodiment, the sensor module comprises a plurality of μLEDs, in particular at least two or three, which are each designed to emit light of a different wavelength, wherein each of the μLEDs is arranged on two of the plurality of contact elements and is electrically connected thereto.

According to at least one embodiment, the integrated circuit is manufactured using CMOS technology and, in particular, is formed by a CMOS (complementary metal-oxide-semiconductor) driver IC. CMOS is a term for semiconductor components in which both p-channel and n-channel field-effect transistors are used on a common substrate.

According to at least one embodiment, the photodetector is formed by a broadband photodiode. The photodetector can be designed accordingly to detect broadband light falling on the detector.

According to at least one embodiment, the photodetector can also be formed by a photodiode array with a plurality of photodiode segments. In addition, a selective light filter can be assigned to each of the photodiode segments or at least some of the photodiode segments, so that only light of a specific wavelength can be detected by means of the photodiode segments in combination with a respective selective light filter. This can, for example, reduce the detection of ambient light and thus noise from the sensor. In at least one embodiment, the size of the light-sensitive area of the photodetector can be a multiple of the distance between two neighboring μLEDs. The same can apply to segments of the photodetector.

According to at least one embodiment, the at least one or the plurality of μLEDs is designed to emit one of green, red or infrared light. In particular, in the case of a plurality of μLEDs, at least one μLED is designed to emit green light, at least one μLED is designed to emit red light, and at least one μLED is designed to emit infrared light. However, the at least one or at least one of the plurality of μLEDs can also be designed to emit blue light, and/or at least one μLED of the plurality of μLEDs can be designed to emit ultraviolet light.

According to at least one embodiment, the at least one μLED comprises a conversion layer by means of which a light of a first wavelength emitted by the μLED is converted into light of a second wavelength different from the first wavelength. For example, white, infrared or broadband light can be generated by a blue-emitting μLED in combination with a conversion layer.

According to at least one embodiment, a plurality of μLEDs are arranged symmetrically around the photodetector on the first main surface. However, it is also conceivable that the μLEDs are arranged next to the photodetector on the first main surface in a partially symmetrical or randomly scattered manner on the first main surface. The number and arrangement of the μLEDs, as well as the number and arrangement of the photodetectors, is arbitrary.

According to at least one embodiment, the sensor module further comprises a plurality of solder balls which are arranged on the second main surface and by means of which the sensor module can be electrically connected. The solder balls can be arranged in the form of a ball grid array on the underside of the sensor module and serve as electrical connections for SMD assembly of the sensor module.

According to at least one embodiment, the sensor module comprises an optical barrier arranged between the photodetector and the at least one μLED on the first main surface. The optical barrier, for example in the form of a rectangular, round or oval frame around the photodetector, preferably projects beyond both the photodetector and the μLED in a direction perpendicular to the first main surface. One reason for using such a barrier is to avoid so-called cross-talks between neighboring components such as μLED and detector.

According to at least one embodiment, the sensor module comprises a carrier substrate on which the semiconductor body is arranged. The carrier substrate can, for example, be a printed circuit board on which the semiconductor body is arranged and to which it is electrically connected.

According to at least one embodiment, the sensor module further comprises at least one bonding wire that electrically connects one of the plurality of contact elements on the first main surface of the semiconductor body to the carrier substrate. In particular, the sensor module comprises a plurality of bonding wires, each of which electrically connects one of the plurality of contact elements on the first main surface of the semiconductor body to the carrier substrate.

According to at least one embodiment, a transparent mold compound is arranged on the first main surface, which covers the photodetector and the at least one μLED. In particular, the transparent mold compound encapsulates the photodetector and the μLED and, in the case of bonding wires, also the bonding wires in order to protect them from external influences and also to improve their aging behavior.

According to at least one embodiment, the at least one, at least one of the plurality, or each of the plurality of μLEDs is formed by a surface emitting μLED. In particular, such a surface-emitting μLED may be formed as a flip chip and arranged on the first main surface such that the light-emitting surface of the μLED faces away from the first main surface.

However, the at least one, at least one of the plurality, or each of the plurality of μLEDs may also be formed by a volume-emitting μLED or an edge-emitting μLED. In particular, the at least one, at least one of the plurality, or each of the plurality of μLEDs may be formed and arranged on the first main surface such that the μLEDs predominantly emit light in a direction away from the first main surface.

In some embodiments, the at least one, at least one of the plurality, or each of the plurality of μLEDs is formed by a sapphire flip chip, a flip chip emitting light through its side surfaces, a surface emitter, a volume emitter, an edge emitter, or by a horizontally emitting μLED chip.

According to at least one embodiment, two or more of the μLEDs can form a pixel, such as an RGB pixel comprising three μLED chips. For example, an RGB pixel can emit light of the colors red, green and blue as well as any mixed colors. In some embodiments, more than three μLEDs can also form a pixel, such as an RGBW pixel comprising four μLED chips. For example, an RGBW pixel can emit light of the colors red, green, blue and white as well as any mixed colors.

Further disclosed is a wearable electronic device, in particular a smartwatch, comprising a sensor module according to at least one of the proposed aspects. The sensor module is preferably integrated into a side of the electronic device facing the skin of a human wearer of the device. In particular, however, the sensor module may occupy only a small area of the side of the electronic device facing the skin and may be virtually invisible to the eye. In some embodiments, the proposed sensor module is used in a wearable device, such as a smartwatch, a blood pressure or pulse monitor and the like, in such a way that the sensor module faces the skin of a user.

In addition, a sensor module according to at least one of the proposed aspects can be used in a display, in particular a μLED display. In particular, the display has a plurality of pixels arranged in rows and columns, each pixel comprising at least one light-emitting component, such as a μLED.

The display can be, for example, the display of a smartphone, a tablet, a laptop, a television, a display of consumer products, a smartwatch or another portable electronic device. The sensor module can be used to record the vital parameters of a user of the display, in particular when the user touches the display in the area of the sensor module.

Each of the pixels can, for example, comprise at least three light-emitting components that are designed to emit light with the colors red, green and blue. Accordingly, the pixels can each be RGB pixels.

At least two μLEDs of the sensor module can, for example, be arranged on the first main surface of the semiconductor body in such a way that the distance between them correlates with the distance between the light-emitting components of the pixels of the display. In other words, the μLEDs of the sensor module thus follow the grid dimension of the display. Thus, instead of a certain number of pixels, the sensor module can be arranged at the corresponding position in the display, and the μLEDs of the sensor module can replace the missing light-emitting pixels of the display.

In some other embodiments, the size of the photodetector may be a multiple of the pixels of the display and located at a position that correlates to the pixels. The photodetector may extend to intermediate pixel spaces.

### BRIEF DESCRIPTION OF THE DRAWINGS

In the following, embodiments of the invention are explained in more detail with reference to the accompanying drawings. They show, in each case schematically,

FIG. 1 a sectional view of a VSM sensor;

FIG. 2 an isometric view of a sensor module according to some aspects of the proposed principle;

FIG. 3A to 3C a top view, an isometric view, and a side view of a sensor module according to some aspects of the proposed principle;

FIG. 4 an isometric view of a further embodiment of a sensor module according to some aspects of the proposed principle;

FIGS. 5A to 5C a top view, an isometric view, and a side view of another embodiment of a sensor module according to some aspects of the proposed principle; and

FIG. 6 an isometric view of a possible use of a sensor module according to some aspects of the proposed principle.

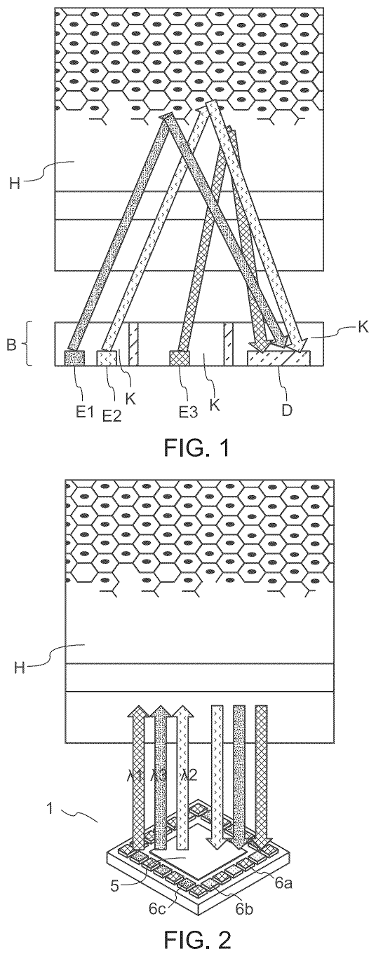

### DETAILED DESCRIPTION

The following embodiments and examples show various aspects and their combinations according to the proposed principle. The embodiments and examples are not always to scale. Likewise, various elements may be shown enlarged or reduced in size in order to emphasize individual aspects. It is understood that the individual aspects and features of the embodiments and examples shown in the figures can be readily combined with each other without affecting the principle of the invention. Some aspects have a regular structure or shape. It should be noted that slight deviations from the ideal shape may occur in practice without, however, contradicting the inventive concept.

In addition, the individual figures, features and aspects are not necessarily shown in the correct size, and the proportions between the individual elements are not necessarily correct. Some aspects and features are emphasized by enlarging them.

However, terms such as “above”, “above”, “below”, “below”, “larger”, “smaller” and the like are shown correctly in relation to the elements in the figures. It is thus possible to deduce such relationships between the elements on the basis of the figures.

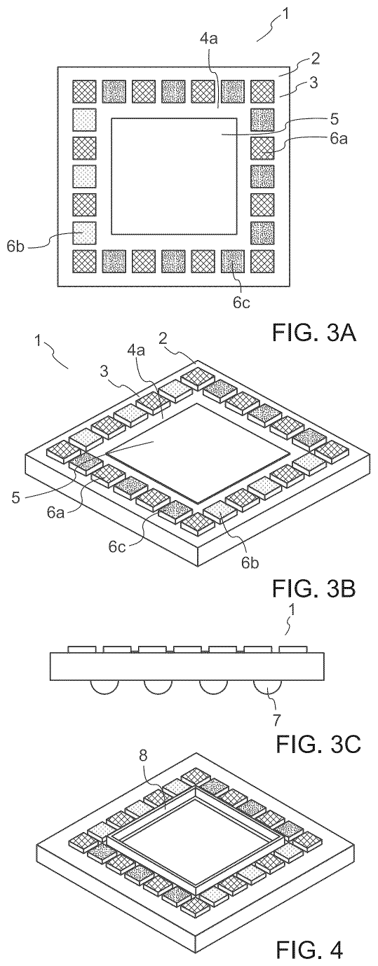

FIG. 2 schematically shows the operation of a sensor module 1 according to some aspects of the proposed principle. As also shown in FIGS. 3A to 3C, the sensor module 1 comprises a semiconductor body 2 in which an integrated circuit 3 and a photodetector 5 are integrated. The photodetector 5 or its photosensitive area is located in a first main surface 4a of the semiconductor body 2. A plurality of μLEDs 6a, 6b, 6c are arranged symmetrically around the photodetector 5 on the first main surface, each of which is designed to emit red, green or infrared light. The μLEDs 6a, 6b, 6c are each arranged on two electrical contact surfaces, not shown here, which are formed on the first main surface 4a of the semiconductor body 2 and by means of which the μLEDs 6a, 6b, 6c are electrically connected to the integrated circuit 3.

In the example shown, 24 μLEDs are arranged in a rectangular shape around a centrally arranged photodetector 5 on the first main surface 4a, whereby twelve first μLEDs 6a are designed to emit green light with a first wavelength λ₁ in the range from 515 nm to 535 nm, six second μLEDs are designed to emit red light with a second wavelength λ₂ in the range from 630 nm to 660 nm, and six third μLEDs are designed to emit infrared light with a third wavelength λ₃ in the range from 830 nm to 870 nm.

To measure a vital parameter, the sensor module 1 is arranged opposite the skin H of a human being, for example, as shown in FIG. 2, so that the first main surface 4a or the photodetector 5 and the μLEDs 6a, 6b, 6c point in the direction of the skin H. By means of the μLEDs, the skin is illuminated with light of the first λ₁, second λ₂ and third λ₃ wavelength and changes in light absorption based on the light reflected back from the skin are measured by means of the photodetector 5. The reflected-back signal detected by the photodetector over a time t consists of light reflections from the tissue or the different skin layers, light reflections from the blood in the veins, which is particularly poor in oxygen, and light reflections from the blood in the arteries, which is particularly rich in oxygen. From the detected signal and in particular from fluctuations or temporal changes in the signal strength, information can then be obtained about, for example, the heart rate or oxygen content of the human being.

FIG. 3C also shows that, according to at least one embodiment, a plurality of solder balls 7 are arranged on a second main surface 4b opposite the first main surface 4a, by means of which the sensor module 1 can be electrically connected. The solder balls can be arranged in the form of a ball grid array on the underside of the sensor module and serve as electrical connections for SMD assembly of the sensor module.

The connections are small solder balls that are placed next to each other in a grid of columns and rows. These beads can be melted in a soldering oven using reflow soldering and then connect to contact pads on a printed circuit board, for example, to which the sensor module is to be attached. This design provides a solution to the problem of accommodating a very large number of connections on one component.

This design also has the advantage that such a sensor module can be removed (desoldered) from the circuit board using hot air, for example, without being damaged, despite being soldered to a printed circuit board. The sensor module can then be stripped of the old solder beads (deballing), cleaned and fitted with new solder beads (reballing) so that it can be reused and soldered onto a new PCB. This technique can also be used to replace a defective sensor module when repairing printed circuit boards.

FIG. 4 shows an isometric view of a further embodiment of a sensor module 1 according to some aspects of the proposed principle. The sensor module 1 additionally has an optical barrier 8 or a frame which is arranged between the photodetector 5 and the μLEDs 6a, 6b, 6c on the first main surface 4a and which is optically non-transparent. The frame 8 encloses the photodetector 5 in the lateral direction and projects vertically beyond both the photodetector 5 and the μLEDs 6a, 6b, 6c. Such an optical barrier 8 reduces external interfering light. In addition, there is no direct line of sight between the photodetector 5 and the μLEDs 6a, 6b, 6c, so that so-called cross talk between the photodetector 5 and the μLEDs 6a, 6b, 6c is prevented.

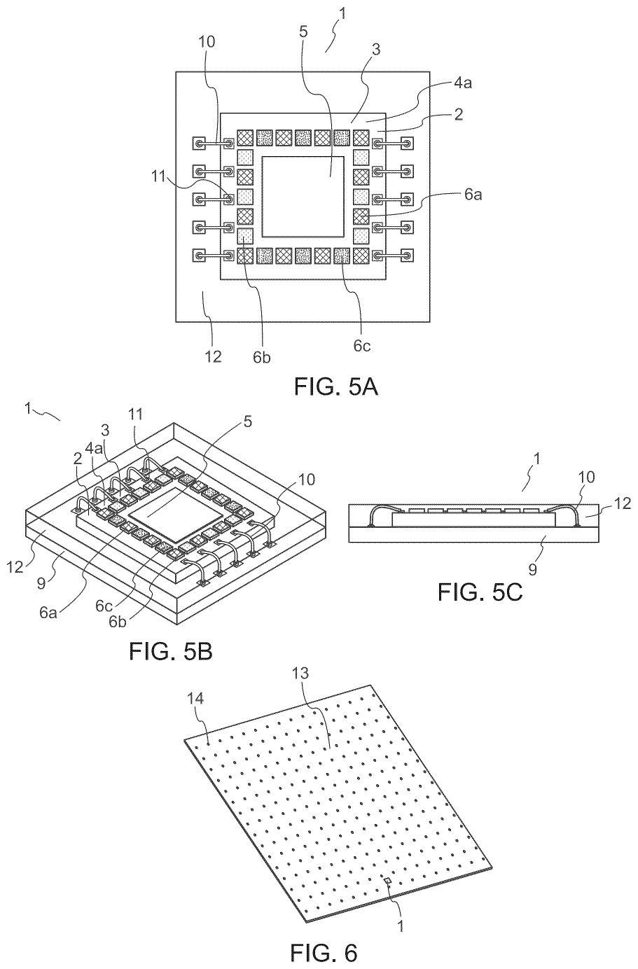

FIGS. 5A to 5C show a further embodiment example of a sensor module 1 according to some aspects of the proposed principle in three different views. Accordingly, the sensor module 1 comprises an additional carrier substrate 9 on which the semiconductor body 2 is arranged. An electrical connection between the carrier substrate 9 and the semiconductor body 2 or the integrated circuit 3 is provided via bonding wires 10, which extend from electrical contact surfaces 11 on the first main surface 4a onto the carrier substrate 9.

The sensor module 1 also has a transparent mold compound 12 that encapsulates the semiconductor body 2, the photodetector 5, the μLEDs 6a, 6b, 6c and the bonding wires 10. As shown in the figures, the mold compound 12 is flush with the carrier substrate 9 on its side surfaces and forms a sealed cuboid together with it.

FIG. 6 shows an isometric view of a possible use of a sensor module 1 according to some aspects of the proposed principle. Accordingly, the sensor module 1 can be installed in a display 13, wherein the display 13 has a plurality of pixels 14 arranged in rows and columns. Each pixel 14 can comprise at least one light-emitting component and, particularly preferably, three light-emitting components, in particular μLEDs. The light-emitting components can also be designed to emit light with the colors red, green and blue and thus form a so-called RGB pixel.

In particular, the μLEDs 6a, 6b, 6c of the sensor module 1 can be arranged on the first main surface 4a of the semiconductor body in such a way that the distance between them correlates with the distance between the individual light-emitting components of the pixels of the display. This makes it possible to integrate the sensor module 1 into the display 13 in a simple manner without it being visible from the outside. Accordingly, the sensor module 1 can be integrated into the display 13 instead of a certain number of pixels 14, and the μLEDs 6a, 6b, 6c of the sensor module 1 can be used on the one hand to detect a vital parameter but also in part to display an image on the display 13.

However, it is also possible to arrange the sensor module 1 only between two pixels 14 of the display 13, as shown as an example in FIG. 6.

### REFERENCE LIST

1 Sensor module
2 Semiconductor body
3 Integrated circuit
4a First main surface
4b Second main surface
5 Photodetector
6a, 6b, 6c μLED
7 Solder ball
8 Optical barrier 8
9 Carrier substrate
10 Bond wire
11 Electrical contact surface
12 Mold compound
13 Display
14 Pixel
E₁, E₂, E₃ Emitter
D Detector
B Component
K Cavity
H Skin
λ₁, λ₂, λ₃ Wavelength

1. A sensor module, in particular for detecting a vital sign parameter, comprising:
a semiconductor body, in particular based on silicon, with a first main surface and a second main surface opposite the first main surface;
an integrated circuit integrated into the semiconductor body;
a photodetector integrated into the semiconductor body and arranged on the first main surface, the photodetector being controllable by means of the integrated circuit;
a plurality of contact elements on the first main surface of the semiconductor body, which are electrically connected to the integrated circuit; and
at least one μLED, wherein the at least one μLED is arranged on two of the plurality of contact elements and is electrically connected thereto.

2. The sensor module according to claim 1, comprising at least two μLEDs, which are each configured to emit light of a different wavelength.

3. The sensor module according to claim 1, wherein the integrated circuit is manufactured using CMOS technology.

4. The sensor module according to claim 1, wherein the photodetector is formed by a broadband photodiode.

5. The sensor module according to claim 1, wherein the photodetector is formed by a photodiode array comprising a plurality of photodiode segments.

6. The sensor module according to claim 5, wherein the photodetector comprises a plurality of selective light filters, each being associated with one of the plurality of photodiode segments.

7. The sensor module according to claim 1, wherein the at least one μLED is configured to emit one of green, red and infrared light.

8. The sensor module according to claim 1, wherein the at least one μLED comprises a conversion layer by means of which a light of a first wavelength emitted by the μLED is converted into light of a second wavelength different from the first wavelength.

9. The sensor module according to claim 2, wherein the at least two μLEDs are arranged symmetrically around the photodetector on the first main surface.

10. The sensor module according to claim 1, further comprising a plurality of solder balls which are arranged on the second main surface and by means of which the sensor module is electrically connectable, in particular to a backplane.

11. The sensor module according to claim 1, further comprising an optical barrier arranged between the photodetector and the at least one μLED on the first main surface.

12. The sensor module according to claim 1, further comprising a carrier substrate on which the semiconductor body is arranged.

13. The sensor module according to claim 12, further comprising at least one bonding wire electrically connecting one of the plurality of contact elements to the carrier substrate.

14. The sensor module according to claim 1, further comprising a transparent mold compound covering the photodetector and the at least one μLED.

15. A wearable electronic device, in particular smartwatch, comprising a sensor module according to claim 1, wherein the sensor module is integrated into a side of the device facing the skin of a human wearer of the device.

16. A use of a sensor module according to claim 1 in a display, in particular μLED display, wherein the display comprises a plurality of pixels arranged in rows and columns, and wherein each pixel comprises at least one light-emitting component.

17. The use of a sensor module according to claim 16, wherein each pixel comprises at least three light-emitting components which are configured to emit light with the colors red, green and blue.

18. The use of a sensor module according to claim 16, wherein at least two μLEDs of the sensor module are arranged on the first main surface of the semiconductor body in such a way that their distance to each other correlates with the distance between the light-emitting components of a pixel of the display.
